(19) United States
(12) Patent Application Publication
(10) Pub. No.: US 2011/0160787 A1
(43) Pub. Date: Jun. 30, 2011

(54) OPTIMIZATION OF AV DELAY USING VENTRICULAR PRESSURE SIGNAL

(75) Inventors: Saul E. Greenhut, Aurora, CO (US); Mustafa Karamanoglu, Fridley, MN (US)

(73) Assignee: Medtronic, Inc.

(21) Appl. No.: 12/751,440

(22) Filed: Mar. 31, 2010

Related U.S. Application Data
(60) Provisional application No. 61/291,038, filed on Dec. 30, 2009.

(51) Int. Cl. A61N 1/365

(52) U.S. Cl. 607/17

(57) ABSTRACT

An implantable medical device system including an intraventricular pressure sensor controls an atrioventricular (AV) delay based on the intraventricular pressure signal. An atrial kick pressure waveform corresponding to active contraction of an atrial chamber is detected from the intraventricular pressure signal. In one embodiment, a time interval corresponding to the atrial kick pressure waveform is measured. An AV delay is set in response to the measured time interval.

### RELATED APPLICATION

The present disclosure claims priority and other benefits from U.S. Provisional Patent Application Ser. No. 61/291,038, filed Dec. 30, 2009, entitled “OPTIMIZATION OF AV DELAY USING VENTRICULAR PRESSURE SIGNAL”, incorporated herein by reference in its entirety

### TECHNICAL FIELD

The disclosure relates generally to implantable cardiac pacing devices and, in particular, to a method and apparatus for optimizing an atrioventricular (AV) delay using a ventricular blood pressure signal.

### BACKGROUND

The timing of atrial activation and resulting contribution of atrial emptying to ventricular filling influences cardiac stroke volume and cardiac output. The term “atrial kick” refers to the active ventricular filling contributed by atrial contraction immediately before ventricular systole. This active filling of the ventricle just before ventricular contraction increases the efficacy of ventricular ejection due to acutely increased ventricular preload. The benefit of atrial kick on ventricular ejection is estimated to account for five to thirty percent of the overall cardiac output. The benefit of the atrial kick can be especially important in patients with compromised ventricular function, e.g. in heart failure. These patients may have implanted pacing devices delivering left ventricular or bi-ventricular pacing to improve ventricular performance. During ventricular pacing, the timing of ventricular activation controlled by an AV delay will directly impact the contribution of atrial kick to cardiac output.

Various methods have been proposed for optimizing an AV delay. Such methods may use timing of cardiac electrical events sensed on ECG or intracardiac electrogram (EGM) signals for determining an optimal AV delay. Other methods may measure hemodynamic parameters and adjust the AV delay to achieve optimal hemodynamic performance of the heart. A need remains, however, for a method and apparatus for optimizing AV delay which promotes optimal benefit of the atrial kick on ventricular performance.

### BRIEF DESCRIPTION OF THE DRAWINGS

FIG. 1 depicts an implantable medical device (IMD) in which monitoring and pacing methods described herein may be implemented.

FIG. 2 is a functional block diagram of one embodiment of the IMD shown in FIG. 1.

FIG. 3 is a flowchart of a method for controlling AV delay using an intraventricular pressure signal.

FIG. 4A is a display of an intraventricular pressure signal and corresponding EGM event markers.

FIG. 4B is an intraventricular pressure signal including a ventricular pressure waveform that begins to rise during the atrial kick waveform.

FIG. 5 is an intraventricular pressure signal shown with an atrial event and a ventricular pacing pulse.

FIG. 6 is a flowchart of one method for adjusting an AV delay based on sensing the atrial kick waveform.

FIG. 7 is a flowchart of an alternative method for optimizing AV delay.

FIG. 8 is a flowchart of a method for controlling AV delay without requiring an atrial EGM/ECG signal.

### DETAILED DESCRIPTION

In the following description, references are made to illustrative embodiments. It is understood that other embodiments may be utilized without departing from the scope of the disclosure. In some instances, for purposes of clarity, identical reference numbers may be used in the drawings to identify similar elements. As used herein, the term “module” refers to an application specific integrated circuit (ASIC), an electronic circuit, a processor (shared, dedicated, or group) and memory that execute one or more software or firmware programs, a combinational logic circuit, or other suitable components that provide the described functionality.

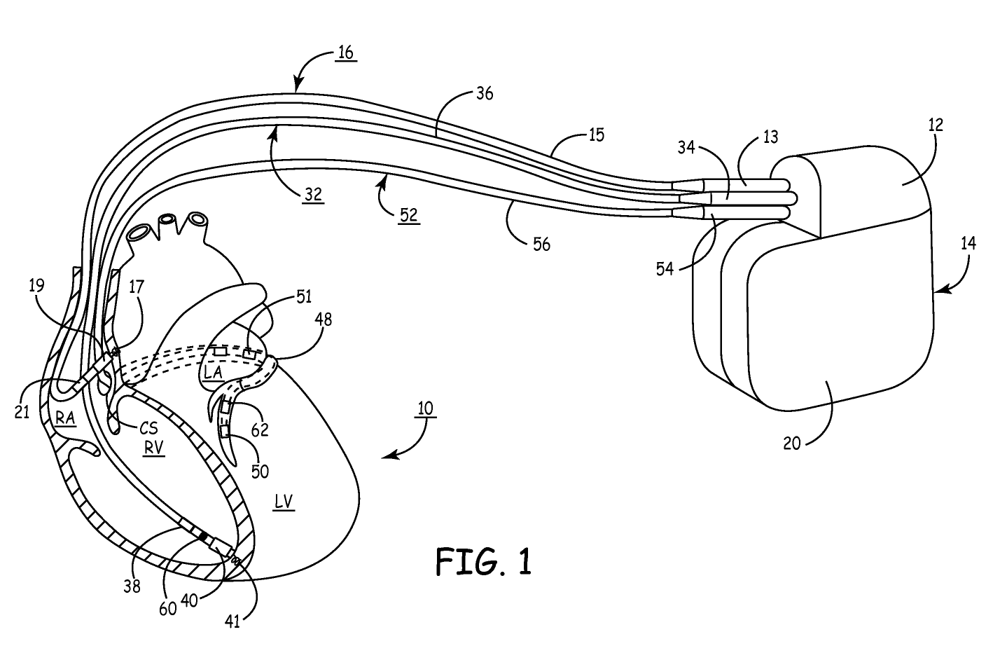

FIG. 1 depicts an implantable medical device (IMD) 14 in which monitoring and pacing methods described herein may be implemented. Various embodiments of the invention may be implemented in numerous types of implantable medical devices capable of sensing cardiac signals and delivering cardiac pacing in at least the ventricular chamber, including various pacemakers and implantable cardioverter defibrillators (ICDs). IMD 14 is provided for sensing intrinsic heart activity and delivering cardiac stimulation pulses in the form of pacing, cardioversion or defibrillation therapy, as appropriate, to one or more heart chambers.

IMD 14 is shown in communication with a patient's heart 10 by way of three leads 16, 32 and 52. The heart 10 is shown in a partially cut-away view illustrating the upper heart chambers, the right atrium (RA) and left atrium (LA), and the lower heart chambers, the right ventricle (RV) and left ventricle (LV), and the coronary sinus (CS) in the right atrium leading into the great cardiac vein 48, which branches to form inferior cardiac veins. Leads 16, 32 and 52 connect IMD 14 with the RA, the RV and the LV, respectively. Each lead has at least one electrical conductor and pace/sense electrode. A remote indifferent can electrode is formed as part of the outer surface of the IMD housing 20. The pace/sense electrodes and the remote indifferent can electrode can be selectively employed to provide a number of unipolar and bipolar pace/sense electrode combinations for pacing and sensing functions.

RA lead 16 is passed through a vein into the RA chamber and may be attached at its distal end to the RA wall using an optional fixation member 17. RA lead 16 is formed with a connector 13 fitting into a connector bore of IMD connector block 12 for electrically coupling RA tip electrode 19 and RA ring electrode 21 to IMD circuitry housed within housing 20 via insulated conductors extending within lead body 15. RA tip electrode 19 and RA ring electrode 21 may be used in a bipolar fashion, or in a unipolar fashion with IMD housing 20, for achieving RA stimulation and sensing of RA EGM signals.

RV lead 32 is passed through the RA into the RV where its distal end, carrying RV tip electrode 40 and RV ring electrode 38 provided for stimulation in the RV and sensing of RV EGM signals, is fixed in place in the RV apex by a distal fixation member 41. RV lead 32 is formed with a connector 34 fitting into a corresponding connector bore of IMD connector block 12. Connector 34 is coupled to electrically insulated conductors within lead body 36 and connected with distal tip electrode 40 and ring electrode 38.

RV lead 32 includes a pressure sensor 60 for monitoring intraventricular blood pressure. As will be described below, the RV pressure signal is used to monitor pressure changes associated with atrial activation and used to optimize an AV pacing delay to promote a maximum benefit of the atrial kick on cardiac output.

A coronary sinus lead 52 may be passed through the RA, into the CS and further into a cardiac vein 48 to extend the distal LV tip electrode 50 and ring electrode 62 alongside the LV chamber to achieve LV stimulation and sensing of LV EGM signals. The LV CS lead 52 is coupled at the proximal end connector 54 into a bore of IMD connector block 12 to provide electrical coupling of conductors extending from electrodes 50 and 62 within lead body 56 to IMD internal circuitry. In some embodiments, LV CS lead 52 could bear a proximal LA pace/sense electrode 51 positioned along CS lead body 56 such that it is disposed proximate the LA for use in stimulating the LA and/or sensing LA EGM signals.

In addition to or in place of the lead-mounted electrodes shown in FIG. 1, IMD 14 may include one or more subcutaneous cardiac sensing electrodes (not shown) formed along the IMD housing 20 or included in the connector block 12. While a particular IMD system with associated leads and electrodes is illustrated in FIG. 1, numerous implantable cardiac pacing system configurations are possible, which may include one or more leads deployed in transvenous, subcutaneous, or epicardial locations or a leadless electrodes incorporated along the IMD housing or connector block.

IMD 14 is shown as a multi-chamber device capable of sensing and stimulation in three or all four heart chambers. It is understood that IMD 14 may be modified to operate as a dual chamber device or a single chamber device, which may or may not have dual chamber sensing capabilities. In the illustrative embodiments described herein, methods for controlling an AV delay generally relate to a pacemaker or ICD having at least dual chamber sensing and pacing in the right atrium and right ventricle. It is contemplated, however, that the methods described, may be adapted for use in a multi-chamber device to control both right and left AV delays or in a device delivering only ventricular pacing and sensing in the atrium and ventricle. In still other embodiments, methods described herein may be implemented in a a single chamber ventricular pacing device or a bi-ventricular pacing device that does not include atrial EGM sensing capabilities.

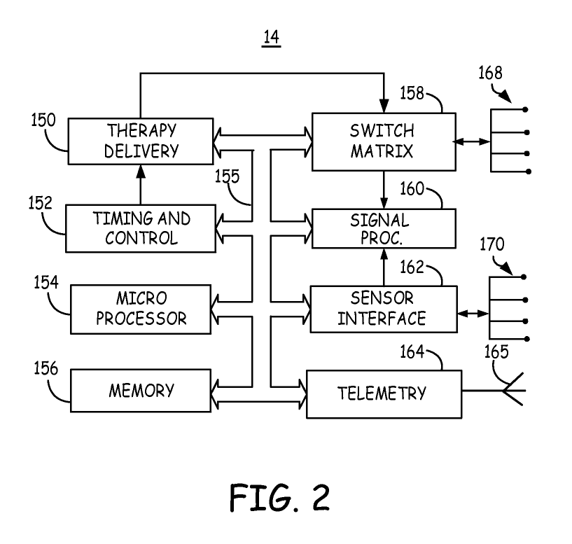

FIG. 2 is a functional block diagram of one embodiment of IMD 14. IMD 10 generally includes timing and control circuitry 152 and an operating system that may employ microprocessor 154 or a digital state machine for timing sensing and therapy delivery functions (when present) in accordance with a programmed operating mode. Microprocessor 154 and associated memory 156 are coupled to the various components of IMD 14 via a data/address bus 155.

IMD 14 may include therapy delivery module 150 for delivering a therapy in response to determining a need for therapy, e.g., based on sensed physiological signals. Therapy delivery module 150 may provide drug or other fluid delivery therapies and/or electrical stimulation therapies. In particular, therapy delivery module includes a pulse generator used to delivery cardiac pacing therapies. Therapies are delivered by module 150 under the control of timing and control circuitry 152.

Therapy delivery module 150 is coupled to two or more electrode terminals 168 via an optional switch matrix 158 for delivering cardiac pacing. Terminals 168 may be coupled to lead connectors providing electrical connection to electrodes incorporated in IMD housing 20 or other lead-based electrodes, e.g., the various electrodes shown in FIG. 1.

Electrode terminals 168 may also be used for receiving cardiac electrical signals through any unipolar or bipolar sensing configuration. Cardiac electrical signals may be monitored for use in diagnosing or managing a patient condition or may be used for determining when a therapy is needed and controlling the timing and delivery of the therapy. Signal processor 160 receives cardiac signals and includes sense amplifiers and may include other signal conditioning circuitry and an analog-to-digital converter. Cardiac electrical signals received from terminals 168, which may be intracardiac EGM signals, far field EGM signals, or subcutaneous ECG signals, are used to detect a need for cardiac pacing and control the delivery of pacing pulses.

IMD 14 is additionally coupled to one or more sensors of physiological signals via sensor terminals 170. Physiological sensors include a pressure sensor 60 as shown in FIG. 1 and may further include other physiological sensors. Physiological sensors may be carried by leads extending from IMD 14, contained inside the IMD or incorporated in or on the IMD housing 20.

Signals received at sensor terminals 170 are received by a sensor interface 62 which provides sensor signals to signal processing circuitry 160. Sensor interface 162 receives the sensor signal and may provide initial amplification, filtering, rectification, or other signal conditioning. Sensor signals are used by signal processor 160 and/or microprocessor 154 for detecting physiological events or conditions. In particular, signals from pressure sensor 60 are processed by signal processor 160 and/or microprocessor 154 for detecting a time corresponding to an atrial kick waveform present on a ventricular pressure signal. This timing is used by microprocessor 154 and timing and control 152 to adjust an AV delay based on the timing of the atrial kick.

The operating system includes associated memory 156 for storing operating algorithms and control parameter values that are used by microprocessor 154. The memory 156 may also be used for storing data compiled from sensed physiological signals and/or relating to device operating history for telemetry out upon receipt of a retrieval or interrogation instruction.

IMD 14 further includes telemetry circuitry 164 and antenna 165. Programming commands or data are transmitted during uplink or downlink telemetry between IMD telemetry circuitry 164 and external telemetry circuitry included in a programmer or monitoring unit.

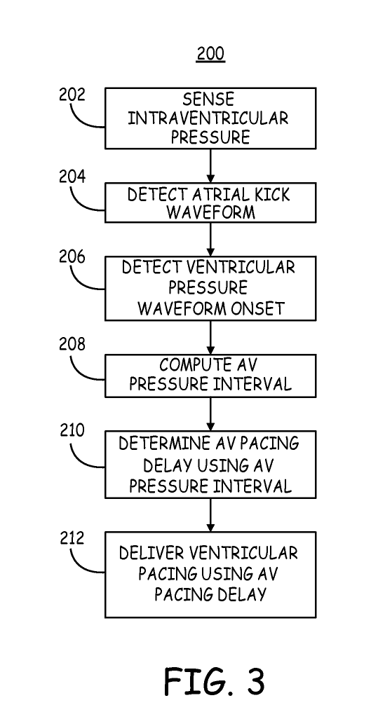

FIG. 3 is a flowchart 200 of a method for controlling AV delay using an intraventricular pressure signal. Flowchart 200, and other flowcharts presented herein, are intended to illustrate the functional operation of the implantable medical device system, and should not be construed as reflective of a specific form of software or hardware necessary to practice the methods described. It is believed that the particular form of software will be determined primarily by the particular system architecture employed in the device system. Providing software, hardware and/or firmware to accomplish the described functionality in the context of any modern implantable medical device system, given the disclosure herein, is within the abilities of one of skill in the art.

Methods described in conjunction with flow charts presented herein may be implemented in a computer-readable medium that includes instructions for causing a programmable processor to carry out the methods described. A “computer-readable medium” includes but is not limited to any volatile or non-volatile media, such as a RAM, ROM, CD-ROM, NVRAM, EEPROM, flash memory, and the like. The instructions may be implemented as one or more software modules, which may be executed by themselves or in combination with other software.

At block 202, an intraventricular pressure signal is sensed. The pressure signal is analyzed at blocks 204 and 206 to detect the atrial kick pressure waveform and the subsequent onset of the ventricular pressure waveform. The pressure signal is sensed in the ventricle but the atrial and ventricular contributions to the pressure signal are referred to as the “atrial kick pressure waveform” and the “ventricular pressure waveform” respectively. The atrial kick pressure waveform, or simply “atrial kick waveform”, is the pressure generation in the ventricle caused by the active contraction of the atrium. The ventricular pressure waveform is the pressure generation in the ventricle caused by active contraction of the ventricle. A time interval between a selected point on the atrial kick waveform and the onset of the ventricular pressure waveform is computed as an AV pressure interval at block 208.

An AV delay is determined at block 210 using the measured AV pressure interval. The AV delay may be computed using additional information such as an estimated electro-mechanical activation delay of the ventricle. In one embodiment, the AV delay is computed such that onset of the ventricular pressure waveform approximately coincides with a peak of the atrial kick waveform. In this way, the beneficial effect of the acutely increased ventricular preload due to atrial kick is promoted. The AV delay may correspond to the time interval between an atrial pacing pulse and a subsequent ventricular pacing pulse, an intrinsic atrial sensed event and a subsequent ventricular pacing pulse, or an atrial kick pressure waveform and a subsequent ventricular pacing pulse. Unique AV delays applied during atrial pacing and during atrial sensing may be determined separately.

The AV delay may be determined at block 210 in a manual or semi-automatic method. A clinician observes a display of the intraventricular pressure signal with visual markers indicating pressure events, such as the atrial kick waveform peak and a ventricular pressure waveform onset. The AV delay may then be adjusted manually or automatically until the clinician observes an optimal intraventricular pressure waveform morphology, e.g. the atrial kick waveform peak and the ventricular pressure onset becoming approximately aligned in time. The current AV delay setting may then be selected for use during ventricular pacing. Ventricular pacing pulses are delivered at block 212 using the selected AV delay.

Alternatively, the AV pressure interval computed at block 208 is used to automatically compute a desired AV delay. The AV delay is automatically adjusted to the computed setting and ventricular pacing pulses are delivered at block 212 using the adjusted delay. Methods for computing the AV delay using the atrial kick waveform may vary between embodiments. Illustrative methods will be described below.

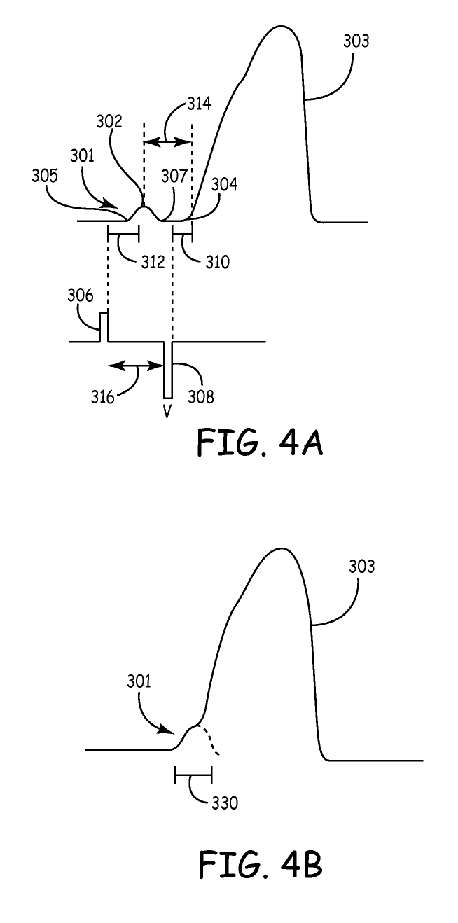

FIG. 4A is a display 300 of an intraventricular pressure signal 303 and corresponding EGM event markers 306 and 308. An atrial kick waveform 301 follows an atrial event 306, which may be a sensed intrinsic P-wave or an atrial pacing pulse and would be labeled accordingly in the display. A ventricular pressure waveform 303 follows a ventricular event 308, which is a ventricular pacing pulse delivered at a current AV delay 316.

Alternatively, for the purposes of making time measurements between the atrial kick waveform 301 and the ventricular pressure waveform 303, the ventricular event 308 may be an intrinsic, sensed R-wave. If the ventricular pressure waveform 303 and the atrial kick waveform 301 are acceptably aligned without ventricular pacing, ventricular pacing may be withheld. In other words if the AV pressure interval is found to be within an acceptable range, and AV conduction is intact, ventricular pacing is not needed. If the ventricular pressure waveform occurs late, after the atrial kick waveform, resulting in a long AV pressure interval, ventricular pacing may be initiated at an optimized AV delay computed using the measured AV pressure interval. Ventricular pacing can be initiated to promote greater ventricular efficacy by optimizing the benefit of the atrial kick. As such, measurements of an AV pressure interval may be used both to determine a need for ventricular pacing as well as for computing an optimal AV delay.

The atrial kick waveform 301 is characterized by a peak pressure 302 occurring at a time interval 312 after the atrial event 306. An onset 304 of the ventricular pressure waveform 303 may be detected based on a threshold crossing of the pressure waveform 303, a threshold of the first time derivative of the pressure waveform (dP/dt), a maximum of the second time derivative of the pressure waveform (d²P/dt²), some other threshold crossing of a high-pass or band-pass filtered pressure signal, or other amplitude or slope change criteria. The ventricular pressure onset 304 follows the ventricular event 308 by a time interval 310, which can be referred to as an “electromechanical delay” in that it is a time interval between an electrical activation of the ventricle and the onset of pressure development by the ventricle.

A clinician viewing a display 300 can adjust the AV delay until the atrial kick peak 302 is approximately aligned with the ventricular pressure waveform onset 304. Alternatively, the AV delay may be adjusted automatically by the pacing device until a time interval 314 between the atrial kick peak 302 and the ventricular waveform onset 304 is within a predefined interval corresponding to an acceptable AV pressure interval. An AV pressure interval may alternatively be measured between atrial kick onset 305 and ventricular pressure waveform onset 304. An optimal AV delay may be computed directly by subtracting the difference between the measured AV pressure interval and a desired AV pressure interval from the current AV delay 316.

In various embodiments, the AV delay may be adjusted until a selected point on the atrial kick waveform 301, such as the onset 305 or peak 302, and a selected point on the ventricular pressure waveform 303 are either aligned or within a predetermined acceptable time interval of each other. A predefined acceptable AV pressure interval will depend on the selected time points being used, e.g. whether the atrial kick waveform onset 301 is being used to measure the start of an AV pressure interval versus the atrial kick waveform peak 302. The AV delay may be set to a time interval that causes the onset of the ventricular pressure waveform 304 and the atrial kick waveform 301 to overlap.

For example, as shown in FIG. 4B, the ventricular pressure waveform 303 begins to rise during the time period 330, which is the expected duration of the atrial kick waveform, between the onset and the end of the downslope of the atrial kick waveform. The greatest benefit on ventricular stroke volume may be achieved when the ventricle begins to contract at or near the peak of the atrial kick waveform.

Referring again to FIG. 4A, the time interval 312 between an atrial event 306 and the atrial kick waveform peak 302 may be measured. The time interval 310 or another time interval corresponding to ventricular electromechanical delay is also measured. A desired AV delay may then be computed as the difference between the time interval 312 and the ventricular electromechanical delay. In this way, a ventricular pacing pulse is delivered after an atrial event 306 but before the atrial kick peak 302 such that the ventricular pressure generation approximately coincides with the peak 302 of the atrial kick waveform.

In another embodiment, the time period from the atrial event 306 to the end 307 of the atrial kick waveform 301, i.e. the end of the downslope portion of the waveform, may be measured and AV delay may be set such that a ventricular pacing pulse is delivered a selected interval before the end 307 of the atrial kick waveform 301 so that ventricular pressure generation will begin during the atrial kick waveform 301.

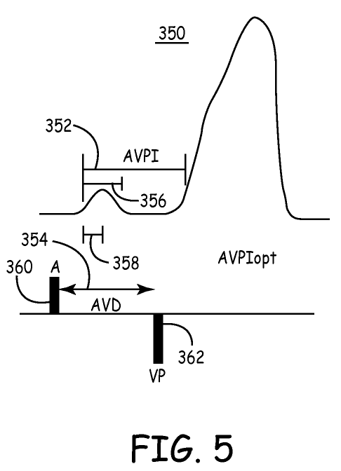

FIG. 5 is an intraventricular pressure signal 350 shown with atrial event 360 and a ventricular pacing pulse 362. The ventricular pacing pulse 362 is delivered at an AV delay (AVD) 354 that results in an AV pressure interval (AVPI) 352. In this example, the AVPI 352 is measured between the onset of the atrial kick waveform 352 and the onset of the ventricular pressure waveform. An optimal AVPI 358 is shown corresponding to the time from the atrial kick onset to the peak of the atrial kick waveform, when it is desirable for ventricular pressure generation to begin. An optimal AV delay is computed as the current AV delay 354 minus the difference between the measured AV pressure interval 352 and the desired AV pressure interval 358.

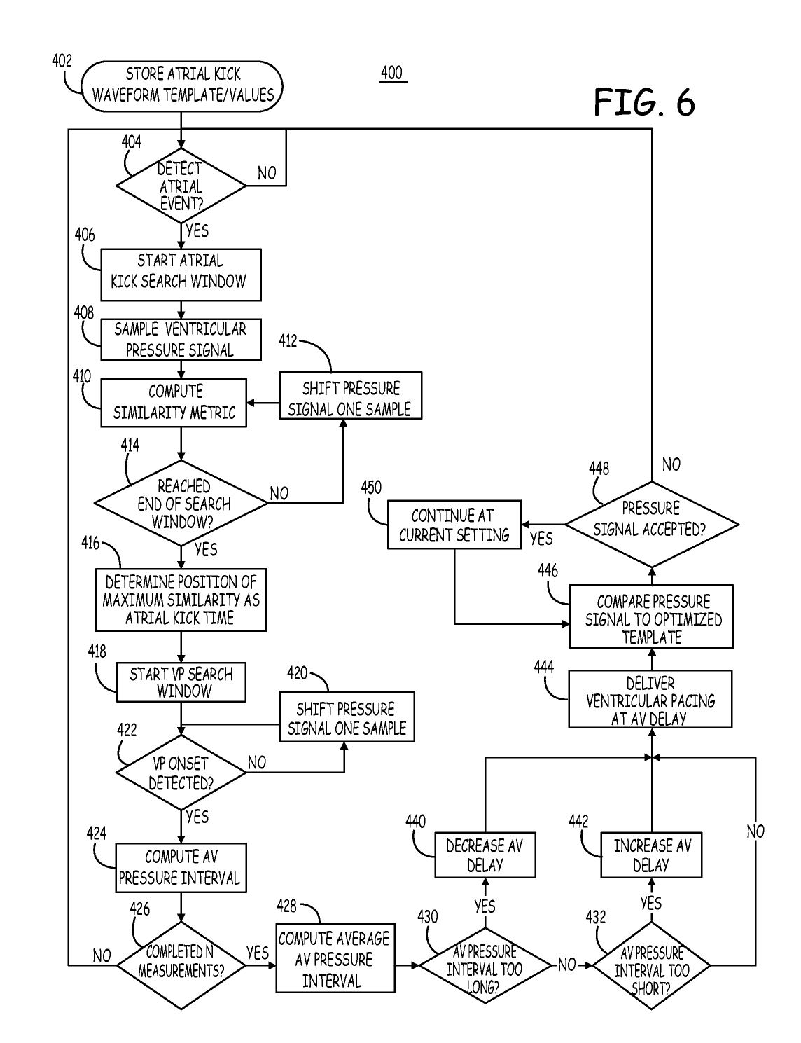

FIG. 6 is a flowchart 400 of one method for adjusting an AV delay based on sensing the atrial kick waveform. At block 402, an atrial kick waveform template or characteristic values of atrial kick waveform features are stored. Values that may be stored include a morphology template, peak amplitude, waveform area, waveform width, waveform slope, or any combination thereof.

Stored template values may be based on clinical data from a population of patients. Alternatively, storing the template or waveform values for an individual patient may include acquiring the ventricular pressure signal, detecting the atrial kick waveform following an atrial pace or sense event, determining the desired waveform features, and averaging these features for multiple atrial kick waveforms to obtain typical values representative of the atrial kick.

For the purposes of generating a morphology template or other characteristic values, the atrial kick waveform may be initially detected based on a timing window between an atrial pace or sense event and a ventricular pace or sense event. It is contemplated that the atrial kick waveform template/values may be acquired and stored when the AV pacing delay is set to a relatively long value such that the atrial kick waveform can be evaluated and characteristic features measured without the influence of active ventricular pressure generation on the pressure signal. The atrial kick template or values may be updated periodically.

In some embodiments, a second template or set of characteristic values may be obtained when the atrial kick and ventricular pressure waveforms are optimally aligned. Optimal alignment may be performed manually or automatically such that the morphology or characteristic values of the pressure signal corresponding to a desired AV pressure interval can be acquired. This optimized template may then be used later to verify optimization of the AV delay.

At block 404, monitoring of the atrial kick waveform for AV delay optimization begins. Upon detecting an atrial event corresponding to electrical depolarization of the atrium, an atrial kick search window is initiated at block 406. The atrial event may be a sensed intrinsic P-wave or an atrial pacing pulse. Method 400 may be performed during atrial sensing to obtain an optimal AV delay setting for use during atrial sensing and repeated during atrial pacing to obtain an optimal AV delay setting for use during atrial pacing. The optimal AV delay during atrial sensing and during atrial pacing may differ.

The atrial kick search window started at block 406 may be defined as a fixed interval of time beginning from the time of the atrial event. The fixed time interval is selected to be long enough to include atrial contraction and relaxation. Alternatively, the atrial kick search window may be a variable time interval beginning at the time of the atrial event and ending upon detecting a subsequent event, such as a ventricular EGM event or the onset of the ventricular pressure waveform.

During the atrial kick search window, the intraventricular pressure signal is sampled at block 408. A similarity metric is computed at block 410 for an interval of consecutive sample points to compare the morphology or other features of the sampled signal to the stored atrial kick template or characteristic values. The interval of consecutive sample points has a duration long enough to capture all or a portion of the atrial kick waveform to allow measurements of the waveform morphology or other characteristic features. Template matching using correlation waveform analysis, wavelet analysis or other methods may be used to compute a similarity metric between the waveform morphology and a stored template. Alternatively, waveform metrics such as amplitude, area, slope, or the like may be compared to stored values to compute a similarity metric. A similarity metric may be computed as a difference between a waveform metric and a stored value, a weighted sum of differences between waveform metrics and corresponding stored values, or other statistical measure of the correlation between stored template values and measured waveform metrics.

If the end of the search window has not been reached at block 414, the interval of consecutive sample points is advanced forward one sample point and the similarity metric is computed again at block 410. This process is repeated until the search window has expired.

At block 416, the similarity metrics computed for each of the intervals of consecutive sample points within the search window are compared and the interval corresponding to the highest similarity metric is identified as the time position of the atrial kick waveform. The start time of the interval may be stored as the time of the atrial kick onset. Alternatively the time point of another characteristic feature, such as the atrial kick peak, end of downslope, or other time point of the atrial kick waveform may be identified within the interval. The stored time point(s) are used to determine an optimal AV delay as will be further described below. The stored time point(s) may be used to define an optimal range of an AV delay, e.g., the onset and end of the atrial kick waveform may be used to determine the shortest and longest optimal AV delay settings, respectively.

At block 418, a ventricular pressure (VP) waveform search window is started and may begin upon a ventricular pace (or sense) event. At block 422, the onset of the ventricular pressure waveform is searched for based on predetermined criteria. For example, the onset of the pressure waveform may be detected as a predetermined threshold crossing of the first time derivative of the pressure signal (dP/dt) or some other high-pass or band-pass filtered signal. In other embodiments the onset of the pressure waveform may be detected as the time of a maximum of the second derivative of the pressure signal (d²P/dt²). The onset may be defined as an inflection point or other slope change that allows identification of the sharp rise in pressure associated with active ventricular pressure generation.

If the onset of the ventricular pressure waveform is not detected at block 422, an interval of pressure signal sample point(s) being evaluated is advanced at block 420 until the ventricular pressure waveform onset is detected.

At block 424, an AV pressure interval is computed. This interval begins at the identified time point of the atrial kick waveform and the time point of the ventricular pressure waveform onset. The interval may optionally be computed for a desired number of cardiac cycles so that the AV pressure interval may be averaged over multiple cardiac cycles. As such, if N measurements have not been completed, as determined at block 426, the process returns to block 404 to repeat the AV pressure interval measurement for the next cardiac cycle.

Once N measurements are complete, an average AV pressure interval is computed at block 428. The AV pressure interval may be compared to an acceptable interval or interval range at blocks 430 and 432. If the measured AV pressure interval is either too long (decision block 430) or too short (decision block 432), the AV delay may be shortened or lengthened at respective blocks 440 and 442. If the AV pressure interval is acceptable, no adjustment is made and the process continues to block 444 where ventricular pacing continues at the current AV delay. The interval or range used at block 430 and 432 to determine if the AV pressure interval is too short or too long will depend on the time points used to compute the AV pressure interval and may vary between patients.

If the interval is too long, the ventricular pressure development may be occurring after the atrial kick and not benefiting from the acutely increased preload. In one embodiment, the AV pressure interval is measured between the maximum peak of the atrial kick waveform and the onset of the ventricular pressure waveform. This interval is desired to be minimized such that the onset of ventricular contraction coincides with the acutely increased preload provided by the atrial kick.

Method 400 may be performed during ventricular pacing at an AV interval that is expected to result in the ventricular pressure waveform onset occurring after the atrial kick waveform. In this way, the atrial kick waveform can be reliably detected without the influence of ventricular pressure generation. The AV pressure interval is measured and if too long, the difference between the measured AV pressure interval and desired interval is used to reduce the current AV delay to an optimal AV delay setting.

If the AV pressure interval is too short, i.e. the ventricular pressure waveform onset is occurring too early relative to the atrial kick peak, the AV delay is lengthened. For example, if the time from the onset of the atrial kick waveform to the onset of the ventricular pressure waveform is very short, the ventricle may be contracting on the upslope of the atrial kick waveform, while the atrium is still contracting. This early ventricular contraction can cause retrograde blood flow from the atrium. In this case the AV delay is lengthened.

In some embodiments, the intraventricular pressure signal analysis may include detecting the atrial kick waveform, then searching for the atrial kick peak and subsequent downslope. If a peak (and optionally downslope) are not detected before the ventricular pressure onset is detected, the AV pressure interval is too short. Pressure signal analysis may include detecting inflection points and slope changes characteristic of the atrial contribution and the ventricular contribution to the intraventricular pressure signal such that when the two signals are merged the onset of ventricular pressure generation during the atrial kick waveform can still be identified.

The adjustments to AV delay at blocks 440 and 442 may be iterative adjustments made in increments or decrements, of approximately 10 or 20 ms at a time for example, until the AV pressure interval falls within an acceptable range. Alternatively, the adjustments to AV delay may be made based on the computed AV pressure interval. For example, if the measured AV pressure interval between an atrial kick peak amplitude and ventricular pressure onset is 30 ms, the current AV delay may be shortened by 30 ms to try to align the atrial kick peak with the ventricular pressure onset. At block 444 ventricular pacing is delivered at the adjusted AV delay. The process may return directly to block 404 to continuously monitor the ventricular pressure signal for optimizing AV delay.

Alternatively, method 400 may optionally include a template matching step at block 446. After adjusting the AV delay, the intraventricular pressure signal may be analyzed and compared to the second pressure signal template stored for an optimized AV pressure interval (at block 402). If the pressure signal approximately matches the optimized AV pressure signal, as determined at block 448, ventricular pacing continues at block 450, at the selected AV delay setting. The pressure signal may be rechecked periodically to verify that the selected setting remains acceptable.

If the pressure signal is not found to be acceptable at block 448 based on template matching analysis, further adjustments to the AV delay can be made by returning to block 404 (or by returning to block 430 or block 432 in an iterative adjustment procedure). The process for computing or adjusting an optimal AV delay may be repeated on a beat-by-beat or less frequent basis for maintaining an optimal AV pressure interval.

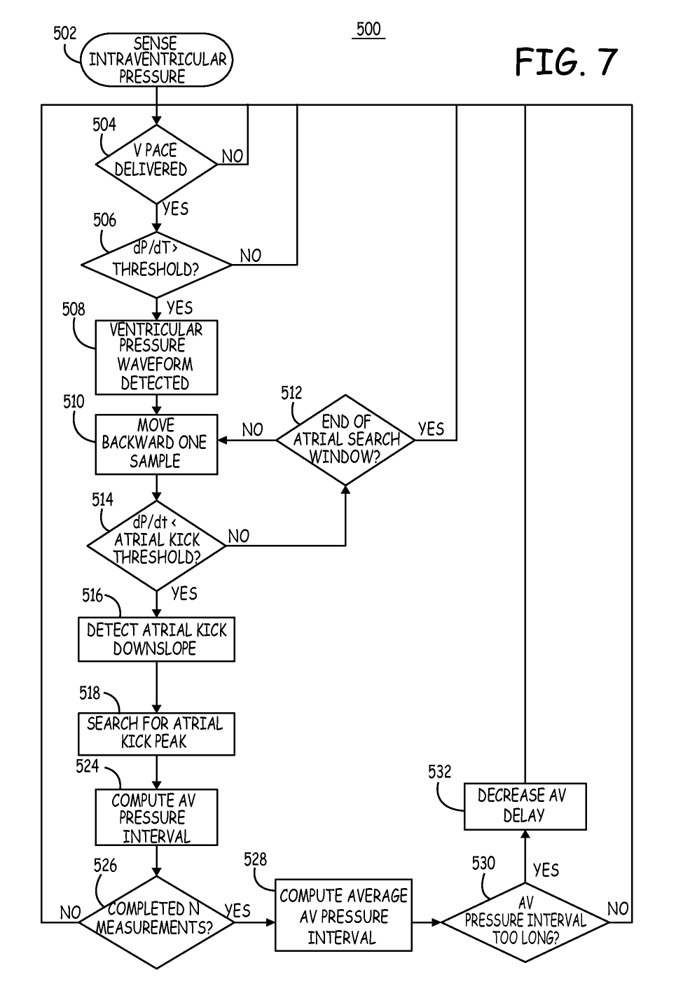

FIG. 7 is a flowchart 500 of an alternative method for optimizing AV delay. At block 502, an intraventricular pressure signal is sensed and stored for signal analysis. Upon delivering a ventricular pacing pulse, as determined at block 504, the intraventricular pressure signal is analyzed at block 506 for detecting the onset of the pressure waveform. The onset may be detected based on a threshold crossing of the first time derivative of the pressure signal (dP/dt) although other criteria, including a threshold crossing of a high-pass or band-pass filtered pressure signal, may also be used. Upon detecting the threshold crossing, yes in block 506, the ventricular pressure waveform onset is detected at block 508.

From the time point of the ventricular pressure waveform onset, the pressure signal is sampled backwards in time beginning at block 510 to search for a downslope of the atrial kick waveform. Various criteria may be used to detect the atrial kick downslope such as detecting a slope change. In one embodiment, if the dP/dt signal is less than an atrial kick downslope threshold, as determined at block 514, the atrial kick waveform is detected at block 516.

If an atrial search window ends before the downslope is detected, the onset of ventricular pressure may be occurring before the downslope, and the AV delay may already be acceptable. The process may then return to block 504 to wait for the next cardiac cycle. The atrial search window may end upon reaching an atrial EGM event, either a sensed atrial P-wave or an atrial pacing pulse, as the pressure signal is sampled going backwards in time.

If the downslope is detected at block 516, the atrial kick peak preceding the downslope is searched for at block 518. Alternatively, an onset of the atrial kick upslope may be detected. An AV pressure interval is computed at block 524 using either the atrial kick peak or the atrial kick onset. This process may be repeated until AV pressure interval measurements for multiple cardiac cycles are obtained (block 526). The N AV pressure interval measurements may be averaged at block 528 and compared to an acceptable interval at block 530. If the AV pressure interval is too long, the AV delay is decreased at block 532. As described previously, the AV delay may be adjusted to produce an AV pressure interval that results in the ventricular pressure onset occurring approximately at the time of the atrial kick peak, or at least during the atrial kick waveform.

The process returns to block 504 to wait for the next ventricular pace event. As described previously, method 500 may be used during ventricular sensing to determine a need for ventricular pacing to optimize the AV delay if intrinsically conducted ventricular activation is occurring too late or too early relative to the atrial kick waveform. The methods described may be performed on a beat-by-beat basis to optimize AV delay, periodically, or in response to detecting altered hemodynamics based on the ventricular pressure signal or other hemodynamic or cardiac function signals.

In variations of the flowchart 500, if an atrial kick downslope is not detected at block 516, a change in slope may be searched for that indicates the ventricular pressure generation onset is occurring during the atrial kick upslope. In this case, the AV delay may be determined to be too short. The AV delay may be lengthened to allow active atrial contraction and the upslope of the atrial pressure generation to be completed before the ventricles begin to contract.

The methods described herein may be repeated at different heart rates (intrinsic or paced) since the optimal AV delay may vary depending on heart rate. As such, if a heart rate change is detected, methods described herein may be performed for detecting the atrial kick waveform and adjusting the AV delay to optimize the AV pressure interval for the existing heart rate.

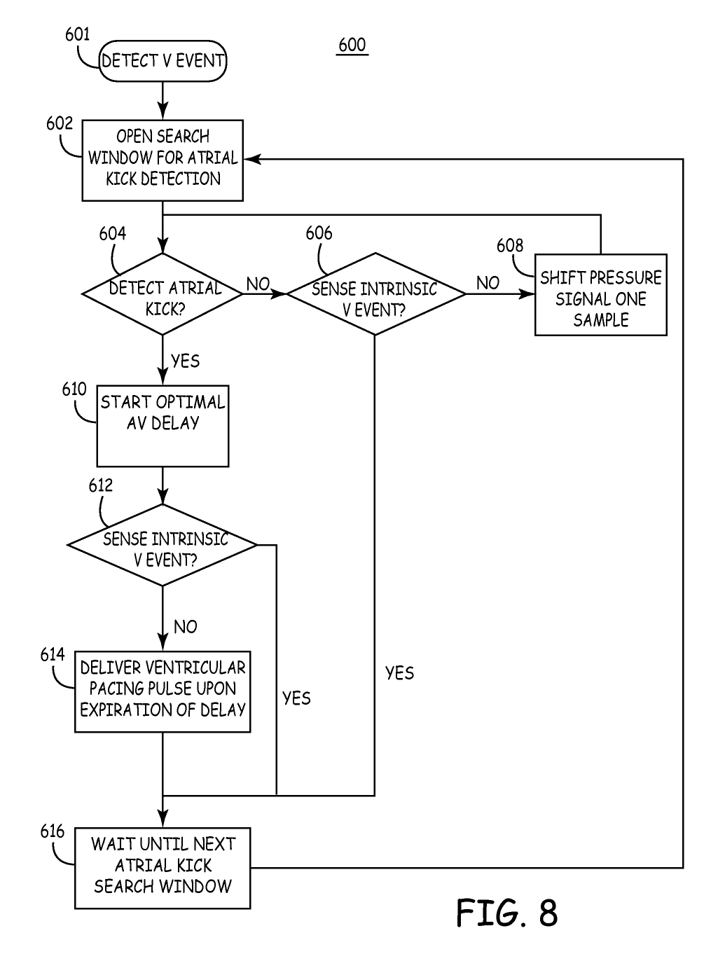

FIG. 8 is a flowchart 600 of a method for controlling AV delay without requiring an atrial EGM/ECG signal. For example, method 600 may be implemented in a single chamber device using a ventricular lead for sensing ventricular EGM signals, delivering ventricular pacing pulses and sensing a ventricular pressure signal. Method 600 could also be implemented in a biventricular device, using right and left ventricular leads without requiring an atrial lead for sensing atrial signals.

At block 601, a ventricular event is detected, which may be a paced or sensed event. An atrial kick search window is started at block 602 after detecting the ventricular event. The atrial kick search window may be initiated after a blanking interval following the ventricular event. The atrial kick search window may be started a predetermined interval after the ventricular event that is heart rate dependent. In other words, the atrial kick search window may be started earlier after a ventricular event during relatively faster heart rates and later after a ventricular event during relatively slower heart rates.

The atrial kick waveform is searched for during the atrial kick window at block 604 using a waveform template comparison or characteristic waveform values in the manner generally described previously. If the atrial kick is not detected, and the next ventricular sensed event is not yet detected (block 606), the process continues to search for the atrial kick waveform by advancing forward one pressure signal within the atrial kick window at block 608

Upon detecting the atrial kick waveform at block 604, an optimal AV delay is started at block 610. The optimal AV delay is set based on the desired AV pressure interval from the time of atrial kick waveform detection to the onset of the ventricular pressure waveform. The optimal delay may be 0 ms such that as soon as the atrial kick waveform is detected, a ventricular pacing pulse is delivered at block 614. Alternatively, the optimal delay may be set to a larger interval as appropriate for timing the ventricular pressure onset to occur during the atrial kick waveform. The optimal delay will depend in part on the detection criteria used to detect the atrial kick waveform. For example, a non-zero delay may be used if the onset of the atrial kick waveform is detected.

If an intrinsic ventricular event is not sensed before the optimal AV delay has expired, a ventricular pacing pulse is delivered at block 614 upon expiration of the optimal AV delay. In this way, the ventricular pacing pulse timing is based on the detection of the atrial kick waveform, without using an atrial EGM/ECG signal, and optimization of the AV delay may occur “on-the-fly” as the atrial kick waveform is detected on each cardiac cycle.

At block 616, the process waits for the time for the next atrial kick search window to be initiated following the ventricular sensed event or pacing pulse. The process then returns to block 602 to start the next atrial kick search window on the next cardiac cycle.

Thus, an implantable medical device system and associated method for controlling an AV delay have been presented in the foregoing description with reference to specific embodiments. It is appreciated that various modifications to the referenced embodiments may be made without departing from the scope of the disclosure as set forth in the following claims.

1. A method for setting an atrioventricular (AV) delay in a cardiac pacing device, the method comprising:
sensing an intraventricular pressure signal;
detecting an atrial kick pressure waveform from the intraventricular pressure signal, the atrial kick pressure waveform corresponding to active contraction of an atrial chamber; and
setting an AV delay in response to detecting the atrial kick pressure waveform.

2. The method of claim 1 further comprising:
measuring a time interval corresponding to the atrial kick pressure waveform; and
setting the AV delay in response to the measured time interval.

3. The method of claim 2 further comprising detecting an onset of a ventricular pressure waveform corresponding to active contraction of a ventricular chamber, wherein the measured time interval comprises an interval from the atrial kick pressure waveform to the ventricular pressure waveform onset.

4. The method of claim 3 wherein the measured time interval comprises an interval beginning from one of an upslope portion of the atrial kick pressure waveform, a peak of the atrial kick pressure waveform, and a downslope portion of the atrial kick waveform.

5. The method of claim 3 further comprising comparing the measured time interval to a predetermined acceptable range of time intervals and adjusting the AV delay to cause the measured time interval to fall within the predetermined acceptable range.

6. The method of claim 1 wherein setting the AV delay comprises setting the AV delay to a value that causes the ventricular pressure waveform onset to occur during the atrial kick waveform.

7. The method of claim 2 wherein the measured time interval comprises an interval from one of an atrial pacing pulse and an atrial P-wave to the atrial kick waveform.

8. The method of claim 7 wherein setting the AV delay comprises setting the AV delay to an interval shorter than the measured interval.

9. The method of claim 1 further comprising:
detecting an atrial upslope portion of the atrial kick waveform;
detecting an onset of a ventricular pressure waveform corresponding to active contraction of a ventricular chamber; and
setting the AV delay to cause the onset of the ventricular pressure waveform to occur after the atrial upslope portion of the atrial kick waveform.

10. The method of claim 2 further comprising determining a need for ventricular pacing in response to the measured interval.

11. An implantable medical device system, comprising:
an intraventricular pressure sensor;
electrodes for sensing cardiac electrical signals and delivering cardiac pacing pulses;
a pulse generator coupled to the electrodes; and
a processor coupled to the pressure sensor, the electrodes, and the pulse generator, the processor configured to:
receive an intraventricular pressure signal from the pressure sensor;
detect an atrial kick pressure waveform from the intraventricular pressure signal, the atrial kick pressure waveform corresponding to active contraction of an atrial chamber; and
set an atrioventricular (AV) delay in response to detecting the atrial kick waveform for controlling ventricular pacing pulses delivered by the pulse generator.

12. The system of claim 11 wherein the processor is further configured to measure a time interval corresponding to the atrial kick pressure waveform and set the AV delay in response to the measured time interval.

13. The system of claim 12 wherein the processor is further configured to detect an onset of a ventricular pressure waveform corresponding to active contraction of a ventricular chamber, and wherein the measured time interval comprises an interval from the atrial kick pressure waveform to the ventricular pressure waveform onset.

14. The system of claim 13 wherein the measured time interval comprises an interval beginning from one of an upslope portion of the atrial kick pressure waveform, a peak of the atrial kick pressure waveform, and a downslope portion of the atrial kick waveform.

15. The system of claim 13 wherein the processor is further configured to compare the measured time interval to a predetermined acceptable range of time intervals and adjust the AV delay to cause the measured time interval to fall within the predetermined acceptable range.

16. The system of claim 11 wherein setting the AV delay comprises setting the AV delay to a value that causes the ventricular pressure waveform onset to occur during the atrial kick waveform.

17. The system of claim 12 wherein the measured time interval comprises an interval from one of an atrial pacing pulse and an atrial P-wave to the atrial kick waveform.

18. The system of claim 17 wherein setting the AV delay comprises setting the AV delay to an interval shorter than the measured interval.

19. The system of claim 11 wherein the processor is further configured to:
detect an atrial upslope portion of the atrial kick waveform;
detect an onset of a ventricular pressure waveform corresponding to active contraction of a ventricular chamber; and
set the AV delay to cause the onset of the ventricular pressure waveform to occur after the atrial upslope portion of the atrial kick waveform.

20. The system of claim 12 wherein the processor is further comprised to determine a need for ventricular pacing in response to the measured interval.

21. A computer-readable medium storing a set of instructions which when implemented in a processor of an implantable medical device system cause the system to:
sense an intraventricular pressure signal;
detect an atrial kick pressure waveform from the intraventricular pressure signal, the atrial kick pressure waveform corresponding to active contraction of an atrial chamber; and
set an atrioventricular (AV) delay in response to detecting the atrial kick pressure waveform.
